(19) United States
(12) Patent Application Publication
(10) Pub. No.: US 2012/0266871 A1
(43) Pub. Date: Oct. 25, 2012

(54) SEAL FOR A DISPENSING APPARATUS

(75) Inventor: Daljit S. Ohbi, Hemel Hempstead (GB)

(73) Assignee: CONSORT MEDICAL PLC, Hemel Hempstead (GB)

(21) Appl. No.: 13/513,609

(22) PCT Filed: Dec. 3, 2010
(86) PCT No.: PCT/GB10/02228

§ 371 Date: Jun. 27, 2012

(30) Foreign Application Priority Data
Dec. 3, 2009 (GB) 0921248.1

(51) Int. Cl. A61M 5/168; B23P 17/00; B29C 45/00; A61M 11/00; A61M 15/00; B65D 47/04; F16K 1/42

(52) U.S. Cl. 128/200.14; 222/544; 222/71; 29/527.1; 251/359; 128/203.15; 604/151; 264/328.17

(57) ABSTRACT

The present invention relates to a seal for a valve for use in a pharmaceutical dispensing device, which seal is formed from an elastomeric composition comprising: (a) one or more elastomers; and (b) a cross-linking agent comprising an aliphatic dialkyl peroxide and/or an aliphatic perketal peroxide.

The present invention relates to a seal material and, in particular, to a seal material comprising a thermoplastic elastomer. The seal may be used for dispensing pressurised fluid in the form of an aerosol. The seal is particularly suitable for use in pharmaceutical dispensing devices such as pressurised metered dose aerosol inhaler devices (pMDIs) and in medical check devices suitable for dispensing a pharmaceutical.

It is known from GB 1201918 for example to provide dispensing apparatus in which pressurised fluid from a pressurised dispensing container is released by a valve in a controlled manner, the valve including elastomeric seals which are annular and which co-operate with a sliding valve stem to open and close fluid ports. FR-A-2,549,568, WO95/02651 and GB 2,148,912 and PCT/GB96/01551 each disclose further examples of such dispensing apparatus.

The required material properties necessary for good seal performance for pharmaceutical applications include: chemical compatibility (swell), tensile strength, permanent compression set, stress relaxation, elastic modulus, regulatory compliance, low permeability to fluids and gases, low levels of extractables and leachables, and stable properties after extraction.

Accordingly, as well as the requirement for good engineering properties, there is a requirement for sanitary properties, including low levels of extractables and leachables, which might otherwise increase impurities of drug products to unacceptable levels, as well as potentially reacting with the drug product, vehicle or excipients. In this connection, products to be dispensed from a pMDI are commonly provided in solution or suspension in a propellant base, this being particularly common in the dispensing of medicinal compounds for inhalation therapy.

The metering valves used in dispensing devices such as pMDIs are typically constructed from a mixture of metal and/or thermoplastic parts and elastomeric rubber parts. The seal itself typically comprises a natural or synthetic elastomer for example, nitrile rubber, neoprene or EPDM.

The production of seals comprising elastomeric materials typically involves steps for the curing/cross-linking of natural and synthetic rubbers. Accelerators are compounds which reduce the time required for curing/cross-linking of natural and synthetic rubbers. Examples include sulphur-based compounds. Accelerators may also act to improve the non-permeability characteristics and other physical properties of the rubber.

The pMDI devices containing propellant and drug mixtures are pressurised at ambient temperatures typically up to 5 bar (500 kPa). Under these conditions the residual by-products from the curing/cross-linking reaction can migrate out and interfere with the drug mechanisms.

Accordingly, in most pharmaceutical applications it is also necessary to extract or wash the cured elastomer in order to remove surface residues and by-products resulting from the cure reaction and moulding process. Examples include ethanol and super-critical fluid extraction. Prolonged extraction times have been found, however, to result in a deterioration in material properties. Moreover, extraction processes add to production costs.

It is an object of the present invention to provide a seal material for a dispensing apparatus which addresses at least some of the problems associated with the prior art.

Accordingly, in a first aspect, the present invention provides seal for a valve for use in a pharmaceutical dispensing device, which seal is formed from an elastomeric composition comprising:
(a) one or more elastomers; and
(b) a cross-linking agent comprising an aliphatic dialkyl peroxide and/or an aliphatic perketal peroxide.

The term seal as used herein is intended to encompass any sealing member or portion thereof present in a pharmaceutical dispensing device, including, but not limited to, gaskets, seats and seals, whether static or dynamic.

It will be appreciated that the seal may be provided as a separate component or may be formed integrally with the valve, i.e. be co-moulded.

The seal according to the present invention is formed from an elastomeric composition, for example a composition comprising a polyolefin elastomer.

The elastomer may comprise a saturated aliphatic polymer. The elastomer preferably comprises one or more ethylenically unsaturated polymers. The unsaturation is preferably provided in the form of alpha-olefin moieties, such as non conjugated double bonds on side-chains of a saturated aliphatic main chain, for example hexa-1,4-diene, and also include unsaturated cyclic monomers such as ethylidenenorbornene, methylidenenorbornene and dicyclopentadiene.

The elastomer is preferably selected from one or more of ethylene propylene (EPM) or ethylene propylene diene (EPDM) rubbers, including derivatives thereof, ethylene vinyl acetate (EAM), chlorinated polyethylene (CM), chlorosulphonated polyethylene (CSM), ethylene acrylate carboxylic acid rubbers (Vamac), fluorocarbon rubbers (FPM), elastomers with unsaturated carbon main chain, polybutadiene (BR), polyisoprene (IR), halogenated butyl rubber (CIIR, BIIR), nitrile rubber (NBR), carboxylated nitrile rubber, hydrogenated nitrile rubber (HNBR), polychloroprene (CR), polyisoprene (IR), styrene butadiene copolymers (SBR). Compositions comprising two or more of the foregoing polymers are also contemplated. Preferably the elastomer is not fluorinated. An example of a fluorocarbon rubber FPM is a fluoroelastomer derived from HXP and polypropylene units having a mooney viscosity measured at 100° C. of between 20 to 120 (ML1) units with a fluorine content of the raw polymer 40% to 70%. An example of the polymer is AFLAS a trademark of the Ashai Glass co, LTD.

EPDM elastomers comprising a terpolymer of ethylene with propylene and a non-conjugated diene providing unsaturation on the side chain are especially preferred. In particular it has been found that the combination of such elastomers with the cross-linking agents described herein provide an improved seal for a dispensing device, for example, having low levels of extractable leachables. Ethylene based terpolymers manufactured using single site metallocene constrained geometery catalyst (CGC) (INSITE process and catalyst technology) are the most preferred in the present invention. The most preferred examples are the Nordel IP and MG for example Nordel IP4520, Nordel IP4640, Nordel IP4725P. Other preferred EPDM elastomers are Vistalon 2504N, Buna G3440, Buna G2440 which are made by the non INSITE technology. Preferred EPM elastomers are Vistalon 404, Vistalon 706.

| | Mooney Viscosity ML | Ethylene, | |
|---|---|---|---|
| | 1 + 4 at | Mass % | |
| | 125° C. ASTM D | ASTM D | ENB, Mass % |
| Elastomer | 1646 MG calculated | 3900 | ASTM D 6047 |
| Nordel IP 4520 | 20 | 50 | 4.9 |
| Nordel IP 4640 | 40 | 55 | 4.9 |
| NORDEL IP 4725P | 25 | 70 | 4.9 |
| Vistalon 2504N | 25 | 56 | 3.8 |
| Buna G3440 | 28 | 48 | 4.1 |
| Buna G2440 | 24 | 51 | 4.3 |
| Vistalon 404 | 28 | 44.5 | — |
| Vistalon 706 | 42 | 65 | — |

It is preferred that the cross-linking agent is of the formula I or II. Formula I shows an aliphatic dialkyl peroxide. Formula II shows an aliphatic perketal peroxide.

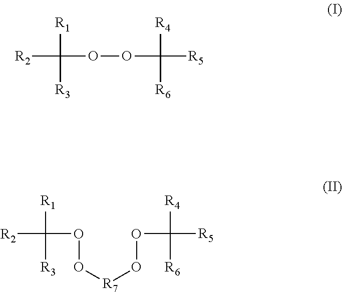

wherein R₁-R₆ are each independently H, a saturated or unsaturated C₁ to C₆ alkyl; and wherein R₇ is a saturated or unsaturated C₁-C₁₈ alkyl, a C₁-C₁₈ alkyl valerate or a cyclic alkyl. It is preferred that R₁-R₆ are saturated. It is preferred that R₇ is a saturated or unsaturated C₁-C₁₈ and, in particular a C1-C18 alkyl valerate, more preferably a C₅ to C₁₅ alkyl valerate.

Crosslinking agents of the aliphatic di-functional di-alkyl type of formula (II) are especially preferred in view of the number of active free-radicals per mole of agent used and their high safe processing temperature. It should be appreciated that an aliphatic di-functional di-alkyl peroxide or an aliphatic perketal peroxide of formula (II) provide four potential active radicals per molecule. However, aliphatic perketal peroxides have a lower safe processing temperature. The peroxide of formula (I) provides only two free radicals per mole.

The present inventors have discovered that the cross-linking agents of the present invention allow for the preparation of a seal that is especially suitable for use in pharmaceutical devices. This is because the cured/crosslinked material produces surprisingly low levels of undesirable leachables and extractables. The levels are sufficiently low that a further extraction step may not necessarily be required. The use of the dialkyl peroxide crosslinking agent is particularly effective for use in preparing an inhaler seal since the high pressure solvents used would otherwise leach undesirable compounds from the seal.

Without wishing to be bound by theory, it is believed that the use of an aliphatic alkyl-group containing peroxide produces impurities which simply remove themselves from the finished material during the curing reaction. In this way there is no need for further processing such as ethanol extraction. Undesirable benzene moieties which are present in dicumyl peroxide, a common curing agent, are thus avoided.

The curing agent is preferably a symmetric peroxide. It is preferably selected from one or more of 2,5-dimethyl-2,5-di-(t-butylperoxy)hexane, 2,5-dimethyl-2,5-di-(t-butylperoxy)hexene-3, 2,5-dimethyl-2,5-di-(t-butylperoxy)hexyne-3, n-butyl-4,4′-di(tert-butylperoxy)valerate, and 1,1′-di(tert-butylperoxy)-3,3,5-trimethylcyclohexane.

The cross-linking agent in the elastomeric composition is preferably in the range of from 0.5% to 10%, more preferably 2% to 4% by weight of the elastomer.

The seal of the present invention may be made from a composition further comprising: (c) a coagent or a crosslink activator.

Coagents and crosslinking activators are well known in the art and are used to increase the crosslinking efficiency of the peroxide. The coagents and crosslink activators in general contain di or polyunsaturation and have readily extractable hydrogen in the alpha position which reacts readily with the peroxide to form coagent free radicals. These radicals are of lower energy and longer life than the free radicals produced by the peroxide. The free radicals formed the coagents prevent the undesirable beta chain scission of the elastomer chain and thereby make the crosslinking reaction predominant over the chain scission reaction. The coagents used in this manner lead to increased tensile strength, hardness and improved compression set resistance. Commonly used coagents and crosslink activators are triallylcyanurate, tri-isoallyl-cyanurate, trimethylol-propanetrimethacrylate, ethylene glycol-dimethacrylate and 1,2-cis-polybutadiene. In a less preferred embodiment, the coagent is m-phenylene dimaleimide. Preferably the composition does not comprise maleimide compounds.

The seal material preferably further comprises a mineral and/or inorganic filler. Mineral fillers are preferable to carbon black in order to minimise the formation of polynuclear aromatic hydrocarbon compounds. Suitable examples include any of magnesium silicate, aluminium silicate, silica, titanium oxide, zinc oxide, calcium carbonate, magnesium oxide magnesium carbonate, magnesium aluminium silicate, aluminium hydroxide, talc, kaolin and clay, including combinations of two or more thereof. Preferably, the filler is or comprises one or more of magnesium silicate, talc, calcined clay, nano particle clays, kaolin and/or amino silane coated clay or clay coated with a titanium or zirconate coupling agent. The filler is typically present in the seal material in an amount of from 1 to 60 wt. %, preferably from 1 to 50 wt. %, more preferably from 1 to 40 wt. %, still more preferably from 1 to 20 wt. %.

The seal may further comprise a process aid, preferably a low molecular weight polyethylene, and, optionally, one or more of a reinforcement agent, a plasticizer, a binder, a stabilizer, a retarder, a bonding agents, an antioxidant, a lubricant, stearic acid, a pigment, a wax, a resin, an antiozonants, a secondary coagent or crosslink activator.

In a second aspect, the present invention provides a valve for use in a pharmaceutical dispensing device having a seal as described above.

In a third aspect, the present invention provides a pharmaceutical dispensing device having a valve as described above. Examples include a pharmaceutical metered dose aerosol inhaler device, a syringe, a vial stopper, a pharmaceutical pump, a peristaltic pump, an auto injector, a medical check valve and a dry powder inhaler.

In most pharmaceutical applications it is also necessary to extract or wash the cured elastomer in order to remove surface residues and by-products resulting from the cure reaction and moulding process. The aforementioned conventional cure/accelerator systems require relatively lengthy extraction times (typically 50 to 70 hours). Prolonged extraction times have been found to result in a deterioration in material properties. The present invention obviates such extraction/washing or at least reduces the time required for extraction/washing.

The polymer blend according to the present may be produced by conventional methods, for example mixing using an intermix twin-screw mixer extruder by injection moulding. Thus, the seal may be produced by a process involving:
(i) forming a composition comprising a mixture of one or more elastomers and a crosslinking agent comprising an aliphatic dialkyl peroxide and/or a aliphatic perketal peroxide;
(ii) initiating a cross-linking reaction in the mixture to form a cross-linked elastomeric composition; and
(iii) either before or after (ii) forming the composition into a seal.

In this process the step of forming the composition into a seal may involve one or more forming techniques such as compression moulding, injection moulding and/or extrusion. The seal material according to the present invention lends itself to commonly used compression, transfer and injection moulding. Injection moulding also results in reduced process waste compared to compression/transfer processes. The seal can also be co-moulded if desired with thermoplastics such as PBT, nylon and/or polyacetal.

The composition may further comprises a coagent or a crosslinking activator. Plasticisers or processing aids or compatibilising agents which are known in the art may also be included in the elastomeric composition.

The seal material crosslinked using the dialkyl peroxide, Luperox F40 (1,3 1.4-bis(tert-butylperoxyisopropyl)benzene) has peroxide related extractable residues in the region 2098 to 4654 ppm (see Table 1). Most of these originate from the aromatic alkyl substituent (diisopropyl benzene) of the Luperox F40 peroxide. In comparison the seal material using an aliphatic dialkyl peroxide according to the present invention is very clean having peroxide related extractible levels of only typically 25 to 228 ppm (by GC-MS)(see Table 2).

As mentioned above, a benefit of using a seal in accordance with the present invention in a pharmaceutical dispensing device is the relatively low levels of leachables and extractables that are present. Thus, while seals prepared according to the present invention may be ethanol extracted (i.e. washed by refluxing ethanol) to reduce the level of any leachable species that could migrate into drug mixtures, this step may be dispensed with if desired. This is in contrast to most conventional thermoset rubbers, which do require an ethanol extraction. As mentioned above, the seal material according to the present invention has peroxide related extractible levels of typically 25 to 228 (see Table 2) (by GC-MS). It will be appreciated that the extractible levels may be further reduced to in the region of 1 to 12 ppm (see Table 3), if required, by performing an extraction step.

The seal according to the present invention may be used in a valve for use in a dispensing device, such as, for example, a nasal, pulmonary or transdermal delivery device. Preferred uses of the seal are in a pressurised pharmaceutical metered dose aerosol inhaler device and in a medical check device suitable for dispensing a pharmaceutical.

The term pharmaceutical as used herein is intended to encompass any pharmaceutical, compound, composition, medicament, agent or product which can be delivered or administered to a human being or animal, for example pharmaceuticals, drugs, biological and medicinal products. Examples include antiallergics, analgesics, bronchodilators, antihistamines, therapeutic proteins and peptides, antitussives, anginal preparations, antibiotics, anti-inflammatory preparations, hormones, or sulfonamides, such as, for example, a vasoconstrictive amine, an enzyme, an alkaloid, or a steroid, including combinations of two or more thereof. In particular, examples include isoproterenol [alpha-(isopropylaminomethyl)protocatechuyl alcohol], phenylephrine, phenylpropanolamine, glucagon, adrenochrome, trypsin, epinephrine, ephedrine, narcotine, codeine, atropine, heparin, morphine, dihydromorphinone, ergotamine, scopolamine, methapyrilene, cyanocobalamin, terbutaline, rimiterol, salbutamol, flunisolide, colchicine, pirbuterol, beclomethasone, orciprenaline, fentanyl, and diamorphine, streptomycin, penicillin, procaine penicillin, tetracycline, chlorotetracycline and hydroxytetracycline, adrenocorticotropic hormone and adrenocortical hormones, such as cortisone, hydrocortisone, hydrocortisone acetate and prednisolone, insulin, cromolyn sodium, and mometasone, including combinations of two or more thereof.

The pharmaceutical may be used as either the free base or as one or more salts conventional in the art, such as, for example, acetate, benzenesulphonate, benzoate, bircarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate, (embonate), pantothenate, phosphate, diphosphate, polygalacturonate, salicylate, stearate, subacetate, succinate, sulphate, tannate, tartrate, and triethiodide, including combinations of two or more thereof. Cationic salts may also be used, for example the alkali metals, e.g. Na and K, and ammonium salts and salts of amines known in the art to be pharmaceutically acceptable, for example glycine, ethylene diamine, choline, diethanolamine, triethanolamine, octadecylamine, diethylamine, triethylamine, 1-amino-2-propanol-amino-2-(hydroxymethyl)propane-1,3-diol, and 1-(3,4-dihydroxyphenyl)-2 isopropylaminoethanol.

The pharmaceutical will typically be one which is suitable for inhalation and may be provided in any suitable form for this purpose, for example as a powder or as a solution or suspension in a solvent or carrier liquid, for example ethanol.

The pharmaceutical may, for example, be one which is suitable for the treatment of asthma. Examples include salbutamol, beclomethasone, salmeterol, fluticasone, formoterol, terbutaline, sodium chromoglycate, budesonide and flunisolide, and physiologically acceptable salts (for example salbutamol sulphate, salmeterol xinafoate, fluticasone propionate, beclomethasone dipropionate, and terbutaline sulphate), solvates and esters, including combinations of two or more thereof. Individual isomers such as, for example, R-salbutamol, may also be used. As will be appreciated, the pharmaceutical may comprise of one or more active ingredients, an example of which is flutiform, and may optionally be provided together with a suitable carrier, for example a liquid carrier. One or more surfactants may be included if desired.

According to a second aspect, the present invention also provides a pharmaceutical dispensing device having a valve as herein described. The pharmaceutical dispensing device may be, for example, a nasal, pulmonary or transdermal delivery device. Preferred devices are a pharmaceutical metered dose aerosol inhaler device and a medical check device.

The present invention also provides a dispensing apparatus for dispensing pressurised fluid comprising a valve body defining a chamber, a valve member extending movably through the chamber and through at least one annular seal co-operating with the valve member and the body to regulate the discharge of fluid, wherein the or at least one of the seals is as herein described with reference to the first aspect of the invention.

Such a device may be used for dispensing medicine, pharmaceuticals, biological agents, drugs and/or products in solution or suspension as herein described.

In a preferred embodiment, the dispensing apparatus comprises a pressurised dispensing container having a valve body provided with two annular valve seals through which a valve member is axially slidable, the seals being disposed at inlet and outlet apertures of a valve chamber so that the valve functions as a metering valve.

The dispensing apparatus as herein described may comprise a pressurised dispensing container operatively connected to the valve body and containing the fluid to be dispensed and a hydrofluorocarbon propellant comprising propellant type 134a or 227. The designation of propellant types referred to in the present application is as specified in British Standard BS4580:1970 “Specification for number designations of organic refrigerants”. Accordingly, propellant 134a is: 1,1,1,2-tetrafluoroethane CH2F—CF3 and propellant 227 is: 1,1,1,2,3,3,3 heptafluoropropane CF3-CHF—CF3.

The fluid to be dispensed typically comprises a liquid or particulate product as a solution or suspension in a carrier liquid. The carrier liquid preferably comprises an alcohol such as ethanol. One or more surfactants may be present.

The present invention will now be described further with reference to the following non-limiting examples.

### EXAMPLES

Seals were prepared by preparing elastomeric compositions, moulding them into the desired shape and crosslinking them to form seals. The below tables indicate the extraction profiles of the various seals made from each of the elastomer compositions used. Table 1 relates to seals cured with Luperox F40 (di(tert-butylperoxyisopropyl)benzene). Tables 2 and 3 relates to seals cured with Trigonox 101.

TABLE 1

| | EPDM Variant | | | | | |
|---|---|---|---|---|---|---|
| | EF269 (F40) | EF269 (F40) | EF287 (F40) | EF287 (F40) | EF281 (F40) | EF281 (F40) |
| Extractable | gaskets | seats (std) | gaskets | seats (std) | gaskets | seats (std) |
| Compound | Tag 398 | Tag 476 | Tag 400 | Tag 488 | Tag 397 | Tag 495 |
| (Units: μg/g) | Vistalon 2504 | Vistalon 2504 | Buna G3440 | Buna G3440 | Nordel IP4520 | Nordel IP4520 |
| 1,3-Bis isopropyl benzene | <2 | 2.20 | 2.02 | 3.91 | <2 | 2.50 |
| 1,4-Bis isopropyl benzene | <2 | 2.27 | 2.08 | 4.05 | <2 | 2.54 |
| 1,3-Isopropenyl acetophenone | 25.3 | 16.8 | 89.0 | 77.4 | 87.1 | 67.5 |
| 1,4-Isopropenyl acetophenone | 9.55 | 6.50 | 37.6 | 29.2 | 38.5 | 26.8 |
| 1,3-Diacetyl benzene | 496 | 585 | 695 | 979 | 456 | 725 |
| 1,4-Diacetyl benzene | 273 | 335 | 373 | 526 | 234 | 392 |
| 1,3-Isopropanol acetophenone | 922 | 900 | 722 | 1134 | 363 | 794 |
| 1,4-Isopropanol acetophenone | 545 | 537 | 432 | 640 | 211 | 450 |
| 1,3-Half peroxide A | 542 | 26.9 | 349 | 12.0 | 92.4 | 4.72 |
| 1,3-Half peroxide B | 1141 | 116 | 980 | 148 | 403 | 82.1 |
| 1,4-Half peroxide A | 166 | 7.58 | 74.4 | 1.97 | 13.1 | <1 |
| 1,4-Half peroxide B | 353 | 51.2 | 317 | 66.4 | 138 | 38.7 |
| 1,3-Peroxide curative | 170 | 1.02 | 177 | 3.74 | 55.2 | <1 |
| 1,4-Peroxide curative | 7.34 | <1 | 9.05 | <1 | 2.3 | <1 |
| TOTAL | 4654 | 2589 | 4259 | 3626 | 2098 | 2589 |

Table 1 shows peroxide related extractables from unextracted EPDM Elastomer cured with Luperox F40. This is a comparative example.

### Key

1,3-Half peroxide A=1-Isopropanol-3-t-butylperoxyisopropyl benzene
1,4-Half peroxide A=1-Isopropanol-4-t-butylperoxyisopropyl benzene
1,3-Half peroxide B=1-Acetyl-3-t-butylperoxyisopropylbenzene
1,4-Half peroxide B=1-Acetyl-4-t-butylperoxyisopropylbenzene
1,3-Peroxide curative=1,3-bis-(tertiary butyl peroxyisopropyl)benzene
1,4-Peroxide curative=1,4-bis-(tertiary butyl peroxyisopropyl)benzene

TABLE 2

| | EPDM Variant | | | | | |
|---|---|---|---|---|---|---|
| Extractable | EF279 (101) | EF279 (101) | EF282 (101) | EF282 (101) | EF288 (101) | EF288 (101) |
| Compound | gaskets | seats (std) | gaskets | seats (std) | gaskets | seats (std) |
| (Units: μg/g) | Tag 450 | Tag 451 | Tag 424 | Tag 478 | Tag 448 | Tag 453 |
| Trigonox 101 | 1.52* | 5.67* | 50.8 | 1.99* | 1.15* | 21.3 |
| *Unknown @ 7.90 min | n/d | 16 | 22 | 62 | n/d | 16 |
| *Unknown @ 7.97 min | n/d | 33 | 32 | 146 | n/d | 49 |
| *Unknown @ 10.64 min | 23 | 4.8 | 43 | 18 | n/d | 7.5 |
| *Unknown @ 20.75 min | n/d | 8.9 | n/d | n/d | n/d | n/d |
| *Unknown @ 22.93 min | n/d | 15 | 21 | n/d | 26 | 25 |
| Isomer A1 @ 21.74 min | n/d | n/d | n/d | n/d | 137 | 142 |
| Isomer A2 @ 22.93 min | n/d | n/d | n/d | n/d | 477 | 458 |
| TOTAL | 25 | 83 | 169 | 228 | 27 | 119 |

*The unknown compounds were not readily identifiable from the GCMS spectra obtained in initial studies but they have been assumed to be peroxide related to present the worst case profile.

Table 2 shows extractables Unextracted Components Cured with Trigonox 101, a cross-linking agent in accordance with the present invention.

Note: to calculate total the values of <x were taken to be equal to x

TABLE 3

| | EPDM Variant | | | | | | | | |
|---|---|---|---|---|---|---|---|---|---|
| Extractable | EF279 (101) | EF279 (101) | EF279 (101) | EF282 (101) | EF282 (101) | EF282 (101) | EF288 (101) | EF288 (101) | EF288 (101) |
| Compound | gaskets | seats (std) | seats (2.5) | gaskets | seats (std) | seats (2.5) | gaskets | seats (std) | seats (2.5) |
| (Units: μg/g) | Tag 444 | Tag 452 | Tag 456 | Tag 395 | Tag 482 | Tag 499 | Tag 449 | Tag 454 | Tag 459 |
| Trigonox 101 | 0.8 | 0.4 | 1.6 | 1.8 | 2.1 | 0.2 | 1.8 | 3.2 | 4.5 |
| Unknown @ | n/d | n/d | n/d | n/d | n/d | n/d | 10.8 | 7.0 | n/d |
| 4.35 min | | | | | | | | | |
| Unknown @ | 1.3 | 1.0 | 1.5 | 1.6 | 2.0 | 1.1 | 1.9 | 2.3 | 2.6 |
| 10.64 min | | | | | | | | | |
| Unknown @ | 2.1 | n/d | n/d | n/d | n/d | n/d | n/d | n/d | n/d |
| 16.79 min | | | | | | | | | |
| TOTAL | 4 | 1 | 3 | 4 | 4 | 1 | 4 | 12 | 7 |

Table 3 shows the extractables from a component cured with Trigonox 101, a cross-linking agent in accordance with the present invention, after a first ethanolic extraction step.

The seal material crosslinked using the dialkyl peroxide, Luperox F40 (1,3 1.4-bis(tert-butylperoxyisopropyl)benzene) has peroxide related extractable residues in the region 2098 to 4654 ppm (see Table 1). Most of these originate from the aromatic alkyl substituent (diisopropyl benzene) of the Luperox F40 peroxide. In comparison the seal material using an aliphatic dialkyl peroxide according to the present invention is very clean having peroxide related extractible levels of only typically 25 to 228 ppm (by GC-MS) (see Table 2).

As mentioned above, and as evidenced in the foregoing examples, a benefit of using a seal in accordance with the present invention in a pharmaceutical dispensing device is the relatively low levels of leachables and extractables that are present. This is in contrast to most conventional thermoset rubbers, which require an ethanol extraction. The seal material according to the present invention has peroxide related extractible levels of typically 25 to 228 (see Table 2) (by GC-MS). It will be appreciated that the extractible levels may be further reduced to in the region of 1 to 12 ppm (see Table 3), if required, by performing an extraction step.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A seal for a valve for use in a pharmaceutical dispensing device, which seal is formed from an elastomeric composition comprising:
(a) one or more elastomers; and
(b) a cross-linking agent comprising an aliphatic dialkyl peroxide and/or an aliphatic perketal peroxide.

2. A seal according to claim 1, wherein the cross-linking agent is of the formula I or II; 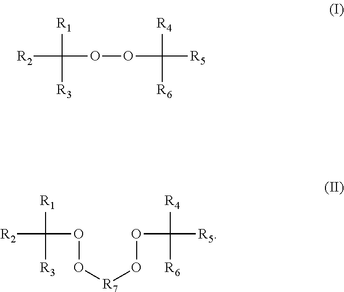
wherein R₁-R₆ are each independently H, a saturated or unsaturated C₁ to C₆ alkyl; and wherein R₇ is a saturated or unsaturated C₁-C₁₈ alkyl, a C₁-C₁₈ alkyl valerate or a cyclic alkyl.

3. A seal as claimed in claim 1, wherein the cross-linking agent is a symmetric peroxide, preferably selected from one or more of:
2,5-dimethyl-2,5-di-(t-butylperoxy)hexane,
2,5-dimethyl-2,5-di-(t-butylperoxy)hexene-3,
2,5-dimethyl-2,5-di-(t-butylperoxy)hexyne-3,
n-butyl-4,4′-di(tert-butylperoxy)valerate, and
1,1′-di(tert-butylperoxy)-3,3,5-trimethylcyclohexane.

4. A seal as claimed in claim 1, wherein the one or more elastomers comprise an ethylenically unsaturated elastomer.

5. A seal as claimed in claim 1, wherein the one or more elastomers comprise one or more of:
ethylene propylene (EPM), ethylene propylene diene (EPDM) rubbers, ethylene vinyl acetate (EAM), chlorinated polyethylene (CM), chlorosulphonated polyethylene (CSM), ethylene acrylate carboxylic acid rubbers (Vamac), fluorocarbon rubbers (FPM), polybutadiene (BR), polyisoprene (IR), halogenated butyl rubber (CIIR, BIIR), nitrile rubber (NBR), carboxylated nitrile rubber, hydrogenated nitrile rubber (HNBR) polychloroprene (CR), polyisoprene (IR) or styrene butadiene copolymers (SBR), including derivatives thereof.

6. A seal as claimed in claim 1, wherein the one or more elastomer comprises at least one of Nordel IP4520, Nordel IP4640, Nordel IP4725P Vistalon 2504N, Buna G3440, Buna G2440, Vistalon 404 and Vistalon 706.

7. A seal according to claim 1, wherein the composition further comprises:
(c) a coagent or a crosslink activator.

8. A seal as claimed in claim 1, wherein the cross-linking agent in the elastomeric composition is in the range of from 0.5 to 10%, more preferably from 2 to 4%.

9. A seal as claimed in claim 1, wherein the seal further includes a mineral filler.

10. A seal as claimed in claim 9, wherein the mineral filler is selected from one or more of magnesium silicate, aluminium silicate, silica, titanium oxide, zinc oxide, calcium carbonate, magnesium oxide magnesium carbonate, magnesium aluminium silicate, aluminium hydroxide, talc, kaolin, clay and amino silane coated clay, nano particle clay or silica.

11. A seal as claimed in claim 1, wherein the seal further includes a process aid selected from the group consisting of a low molecular weight polyethylene, stearic acid or a organic or non-organic stearate.

12. A seal as claimed in claim 1, further comprising one or more of a reinforcement agent, a plasticizer, a binder, a stabilizer, a retarder, a bonding agents, an antioxidant, a lubricant, a pigment, a wax, a resin, an antiozonants, a secondary coagent or a secondary crosslink activator.

13. A valve for use in a pharmaceutical dispensing device having a seal as defined in claim 1.

14. A pharmaceutical dispensing device having a valve as claimed in claim 13.

15. A pharmaceutical dispensing device as claimed in claim 14 which is a pharmaceutical metered dose aerosol inhaler device, a syringe, a vial stopper, a pharmaceutical pump, a peristaltic pump, an auto injector, a medical check valve or a dry powder inhaler.

16. A dispensing apparatus for dispensing pressurised fluid comprising a valve body defining a chamber, a valve member extending movably through the chamber and through at least one annular seal co-operating with the valve member and the body to regulate the discharge of fluid, wherein the annular seal is as defined in claim 1.

17. A dispensing apparatus which comprises a pressurised dispensing container having a valve body provided with two annular valve seals through which a valve member is axially slidable, said seals being disposed at inlet and outlet apertures of a valve chamber so that the valve functions as a metering valve, wherein at least one of the annular valve seals is as defined in claim 1.

18. A dispensing apparatus as claimed in claim 16, comprising a pressurised dispensing container operatively connected to the valve body and containing the fluid to be dispensed and a hydrofluorocarbon propellant comprising propellant type 134a or 227.

19. A dispensing apparatus as claimed in claim 16, wherein the fluid to be dispensed comprises a liquid or particulate product as a solution or suspension in a carrier liquid comprising alcohol.

20. A process for the preparation of a seal for a valve for use in a pharmaceutical dispensing device, the process comprising:
(i) forming a composition comprising a mixture of one or more elastomers and a crosslinking agent comprising a aliphatic dialkyl peroxide and/or an aliphatic perketal;
(ii) initiating a cross-linking reaction in the mixture to form a cross-linked elastomeric composition; and
(iii) either before or after (ii) forming the composition into a seal.

21. A process according to claim 20, wherein the composition further comprises a coagent or a crosslinking activator for the crosslinking agent.

22. A process as claimed in claim 20, wherein the step of forming the composition into a seal involves one or more forming techniques selected from transfer moulding, compression moulding, injection moulding and extrusion.

23. A process as claimed in claim 20, wherein the process further involves a step of washing and/or extraction of the seals using solvents and/or supercritical fluids.
